(19) United States
(12) Patent Application Publication
(10) Pub. No.: US 2025/0302419 A1
(43) Pub. Date: Oct. 2, 2025

(54) METHOD FOR SCATTERED-RADIATION CORRECTION AND APPARATUS

(71) Applicant: Siemens Healthineers AG, Forchheim (DE)

(72) Inventors: Philipp Roser, Erlangen (DE); Philipp Bernhardt, Forchheim (DE); Richard Obler, Erlangen (DE)

(21) Appl. No.: 19/088,046

(22) Filed: Mar. 24, 2025

(30) Foreign Application Priority Data
Mar. 27, 2024 (DE) 102024202913.0

(51) Int. Cl. A61B 6/00; A61B 6/06

(52) U.S. Cl. CPC A61B 6/5282; A61B 6/06

(57) ABSTRACT

For particularly rapid and effective scattered-radiation correction of 2D X-ray images, a method is provided for scattered-radiation correction for an X-ray image or a series of X-ray images, which images may be acquired by an acquisition system having an X-ray source, an X-ray detector, and a collimator that shapes the primary X-ray radiation. The method includes: providing the X-ray image acquired in a first setting of the collimator in which setting the object under examination is illuminated; providing an auxiliary X-ray image acquired in a second setting of the collimator in which second setting are illuminated, in particular solely one or more sub-regions of the X-ray detector; subtracting the auxiliary X-ray image from the X-ray image; determining a scattered-radiation correction using image data from the X-ray detector obtained from the subtraction at least in the region of the sub-regions; and correcting the X-ray image using the determined scattered-radiation correction.

The present patent document claims the benefit of German Patent Application No. 10 2024 202 913.0, filed Mar. 27, 2024, which is hereby incorporated by reference in its entirety.

### TECHNICAL FIELD

The disclosure relates to a method for scattered-radiation correction for a (2D) X-ray image or a series of X-ray images, to a method for scattered-radiation correction for a, e.g., two-dimensional (2D), X-ray image or a series of X-ray images, and to an apparatus for performing such a method.

### BACKGROUND

Scattered radiation is a known and common problem in medical X-ray imaging that impairs the imaging. In three-dimensional (3D) imaging, for and during interventional procedures and examinations, (e.g., in DynaCT), scatter correction is imperative to achieving a reasonable image quality. Also, in 2D imaging, however, during surgical interventions such as fluoroscopy or digital subtraction angiography (DSA), scatter has a negative impact on the perceived and quantitative image quality. To begin, the low-frequency component of the scatter signal acts as an offset to the image intensity, impairs the log normalization, and may reduce the contrast in the native imaging. Additionally, scatter contravenes the normally used assumptions for DSA or roadmap imaging, which in turn may lead to artifacts in the contrasted vessels (e.g., vessels appear white instead of black in DSA).

Apart from using anti-scatter grids to block the scattered radiation, there is rarely any specific scatter correction in 2D imaging. Possible approaches generally have major disadvantages. Extending the use of scatter kernels (as in 3D imaging) from 3D to 2D involves a large amount of effort because of the far larger parameter space (SID, tube voltage, collimation, etc.). Supervised training of AI is also time-consuming, primary modulation needs an additional primary modulator, which is located in the beam path, and unsharp masking may produce large errors.

In one example, scattered radiation in X-ray based medical imaging is estimated and interpolated using image data that was acquired outside the illuminated area, i.e., in the collimator shadow. This approach only works, however, if the collimator shadow is sufficiently large.

### SUMMARY AND DESCRIPTION

An object of the present disclosure is to provide a method for scattered-radiation correction for a (2D) X-ray image, wherein the method allows for rapid and low-effort scattered-radiation correction even for a series of X-ray images. It is also an object of the disclosure to provide an X-ray device suitable for performing the method.

The scope of the present disclosure is defined solely by the appended claims and is not affected to any degree by the statements within this summary. The present embodiments may obviate one or more of the drawbacks or limitations in the related art.

A method for scattered-radiation correction for an X-ray image or a series of X-ray images of an object under examination is disclosed, wherein images may be acquired by an acquisition system having an X-ray source, an X-ray detector, and a collimator that shapes the primary X-ray radiation. The method includes providing the X-ray image, which was acquired in a first setting of the collimator in which the object under examination is illuminated. The method further includes providing an auxiliary X-ray image, which was acquired in a second setting of the collimator in which solely one or more sub-regions of the X-ray detector are illuminated, each of which has a very small area compared with the total area of the X-ray detector. The method further includes subtracting at least in part the image data of the auxiliary X-ray image from the image data of the X-ray image. The method further includes determining a scattered-radiation correction using image data from the X-ray detector obtained from the subtraction at least in the region of the sub-regions. The method further includes correcting the X-ray image using the determined scattered-radiation correction. For example, the object under examination may include an organ to be imaged, part of an organ or body part, or a structure to be imaged, and/or an instrument introduced into the body in the case of interventional procedures. The X-ray image(s) may be two-dimensional projection images. In the subtraction, the image data that was acquired from corresponding or identical image pixels of the X-ray detector is deducted one from the other, prior to which a suitable adjustment may be made to account for X-ray parameters such as the X-ray dose.

A further method for scattered-radiation correction for a (e.g., 2D) X-ray image or a series of X-ray images includes setting a second setting of the collimator so as to illuminate one or more sub-regions of the X-ray detector, each of which has a small area compared with the total area of the X-ray detector (e.g., 1/50^{th} or 1/100^{th} of the total area), and acquiring an auxiliary X-ray image. The method further includes setting a first setting of the collimator so as to illuminate an object under examination, and acquiring an X-ray image. The method further includes subtracting at least in part the image data of the auxiliary X-ray image from the image data of the X-ray image. The method further includes determining a scattered-radiation correction using image data from the X-ray detector obtained from the subtraction at least in the region of the sub-regions. The method further includes correcting the X-ray image using the determined scattered-radiation correction.

By virtue of the method(s), it is possible, in particular for 2D imaging and for series of 2D X-ray images, to provide and perform precise, effective, and rapid scattered-radiation correction that may also be adapted rapidly and easily to changes (e.g., in the angulation or the examination region). The scattered-radiation correction does not require any additional hardware and may be carried out with a very low additional X-ray dose. The computing effort is also kept low. The method may be inserted rapidly between two X-ray images, for example, during live X-ray imaging, by rapid “artificial” inward and outward collimation and acquisition of the auxiliary X-ray image. This may even be performed so rapidly between two fluoroscopy X-ray images that the medical personnel barely notice it. For the patient, the method means no additional load. The quality of the X-ray images may be improved significantly, thereby improving both the diagnosis and the implementation of interventional procedures under X-ray monitoring (e.g., fluoroscopy).

According to one embodiment, before the subtraction, the auxiliary X-ray image is pre-corrected for scattered radiation using image data from the X-ray detector obtained in the collimator shadow. A particularly high image quality is possible by combining the original method with further image quality improvements, for example, a scattered-radiation correction as described above. In this example, data from at least one region of the X-ray image obscured by the collimator (i.e., the collimator shadow), (e.g., in the edge regions of the X-ray image), is used to determine a scattered-radiation correction, because in the collimator shadow no image data may arise from direct X-ray radiation.

According to a further embodiment, a multiplicity of sub-regions are illuminated during the acquisition of the auxiliary X-ray image, wherein each subregion has a small area compared with the total area of the X-ray detector (e.g., 1/50^{th} or 1/100^{th} of the total area). Instead of just one small area, in this case a multiplicity of small areas are illuminated, in particular also sub-regions distributed over the entire area of the X-ray detector, for instance evenly distributed in the manner of an array. It is thereby possible to carry out after a subtraction an even better determination of a scattered-radiation distribution over the entire X-ray image. From the scattered-radiation distribution ascertained in the sub-regions by the subtraction, it is then possible to estimate or calculate, for instance by averaging or interpolation, the scattered radiation in the regions that were not exposed directly in the auxiliary X-ray image. Algorithms or even machine learning methods may be used for this purpose.

According to a further embodiment, the maximum area of individual sub-regions is at most 1/50 or at most 1/100 of the total area of the X-ray detector. The small area of the sub-regions may be much smaller still than these values (e.g., at most 1/500, at most 1/1000, at most 1/5000, or at most 1/10000 of the total area of the X-ray detector). The sub-regions may also be formed as very narrow strips, similar to the case of slot scan technology, for instance at most just a few millimeters wide. Given such a small area, as a result of the narrow collimation, almost no image data is produced by scattered radiation in the sub-region exposed to the primary beam, and therefore subtracting the image data of the exposed sub-region of the auxiliary X-ray image from the image data of the X-ray image results in a very exact representation of the scattered-radiation component of the X-ray image. Should a very small amount of scattered radiation arise nonetheless in the exposed sub-region, this may be removed before the subtraction by alternative scattered-radiation corrections.

According to a further embodiment, an X-ray dose during the acquisition of the auxiliary X-ray image is 1/10th or less of the X-ray dose during the acquisition of the X-ray image. This results in a negligible radiation load for the patient, for instance a maximum of 1/500 of a normal X-ray image ( 1/50× 1/10).

According to a further embodiment, a series of X-ray images is acquired in succession and corrected by the method. For instance, a plurality, or large number, of X-ray images may be corrected by the scattered-radiation correction ascertained by the method. This may be carried out, for example, when no change occurs (e.g., in the X-ray parameters, or movement of the object under examination). If numerous or regular changes occur, a re-acquisition of an auxiliary X-ray image may be carried out each time and used to repeat the method. As this results in little additional X-ray dose and the method may be performed very rapidly (merely a brief inward and outward collimation with image acquisition), this produces negligible disruption to the process.

According to a further embodiment, at least one intermediate auxiliary X-ray image is provided, which was acquired in a third setting of the collimator, in which third setting are illuminated one or more sub-regions, wherein each sub-region has an area that in size is between the total area of the first setting and the small area of the second setting. The intermediate auxiliary X-ray image is used to refine the determined scattered-radiation correction. By virtue of an intermediate auxiliary X-ray image of this type, the area of which that is exposed to the primary radiation is larger than the small area of the second setting, (e.g., 2 or 4 times larger), but smaller than the area of the normal X-ray image, further information about the scattered radiation may be ascertained and used. Whereas the contribution of the scattered radiation to the image data on the area affected by the primary beam is negligible in the case of the small area of the second setting, the area will be larger for the intermediate auxiliary X-ray image, and therefore, for example, data may be used here for interpolating the scattered-radiation contribution.

In another example, further intermediate auxiliary X-ray images may be provided and used for refining the determined scattered radiation. These further intermediate auxiliary X-ray images may be acquired in further settings of the collimator in which sub-regions are illuminated, wherein each sub-region has an area size between the total area of the first setting and the small area of the second setting. This process may improve even further the interpolation or estimate. For instance, the sub-regions affected by the primary radiation may each be enlarged, and a plurality of intermediate auxiliary X-ray images acquired in each case, for example, 2×, 4×, 8×, 16×, etc. Then, the influence of the scattered radiation may be ascertained (e.g., calculated, interpolated, extrapolated, or simulated) from the corresponding intermediate auxiliary X-ray images and the auxiliary X-ray image. The accuracy of the estimate of the scattered radiation may also be improved from the variation in the scattered radiation in the ever-smaller collimator shadow and from the change in intensity for the region, which is irradiated equally each time.

The disclosure also relates to a medical X-ray device for performing an above-described method. The medical X-ray device includes an acquisition system for acquiring X-ray images that has an X-ray detector, an X-ray source for emitting primary X-ray radiation, and a beam-shaping collimator configured to illuminate sub-regions of very small area. The medical X-ray device further includes a control unit for controlling the X-ray device. The medical X-ray device further includes a processing unit for subtracting the auxiliary X-ray image from the X-ray image, for determining a scattered-radiation correction using image data obtained from the subtraction at least in the region of the sub-regions, and for performing the scattered-radiation correction of X-ray images. For example, the X-ray device may be a C-arm X-ray device, wherein the X-ray detector and the X-ray source are arranged on an adjustable C-arm. The collimator is arranged, for instance in the region of the X-ray source, in such a way that using one or more shields or plates may shape the primary X-ray beam in terms of the area onto which the beam shines.

The disclosure and further advantageous embodiments are described in greater detail below with reference to schematically represented embodiments in the drawings, without thereby limiting the disclosure to these embodiments.

### BRIEF DESCRIPTION OF THE DRAWINGS

FIG. 1 depicts an example of a sequence of acts of a method for scattered-radiation correction for a 2D X-ray image.

FIG. 2 depicts an example of a further sequence of acts of a method for scattered-radiation correction for a 2D X-ray image.

FIG. 3 depicts an example of an X-ray image.

FIG. 4 depicts an example of an auxiliary X-ray image.

FIG. 5 depicts an example of a further auxiliary X-ray image.

FIG. 6 depicts an example of a further sequence of acts of a method for scattered-radiation correction for a 2D X-ray image.

FIG. 7 depicts an example of an X-ray device for performing the method.

FIG. 8 depicts an example of a sequence of a method for scattered-radiation correction.

### DETAILED DESCRIPTION

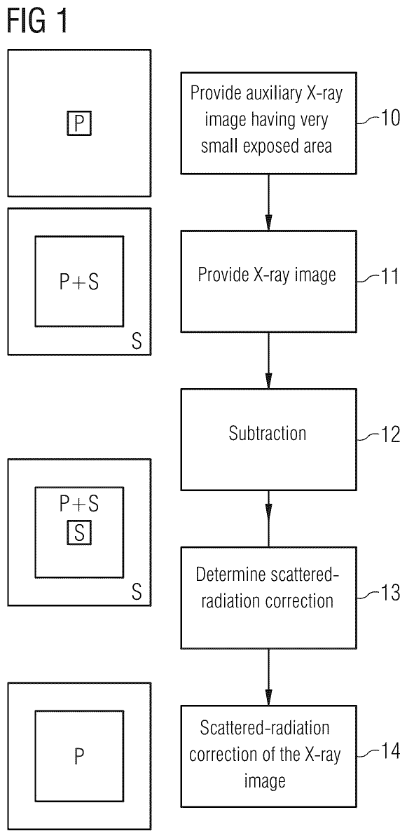

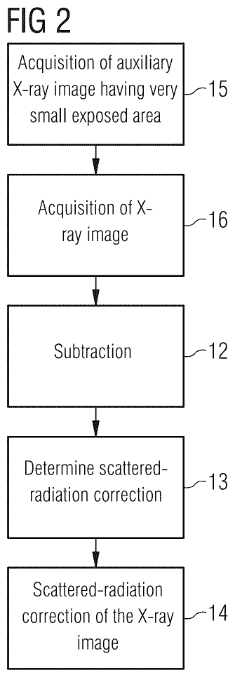

FIGS. 1 and 2 show acts of methods for scattered-radiation correction for a (2D) X-ray image or a series of X-ray images, which methods constitute a rapid and low-effort option for scattered-radiation correction of 2D X-ray images including during interventional procedures under X-ray monitoring. In act 10 (FIG. 1), an auxiliary X-ray image is provided. The auxiliary X-ray image is acquired in a second setting of the collimator, in which setting are illuminated one (or more) sub-regions T of the X-ray detector, each of which has a very small area compared with the total area of the X-ray detector.

FIG. 4 shows a corresponding schematic acquired auxiliary X-ray image 20, which illuminates a sub-region T (situated here by way of example in the center of the X-ray detector) of very small area, so that in the sub-region T affected by the primary radiation, scarcely any or no scattered radiation contributes to the acquired image data, and only a primary beam component P of the image data is present (this is known from the slot scan method, for example). The sub-region T has here, for example, an area of at most 1/50 of the total area of the X-ray detector, in particular also much smaller, for instance with a width of a few pixels or a few millimeters. It is also possible to illuminate two or more sub-regions T (not contiguous), for instance a multiplicity of sub-regions. For example, see a further schematic acquired auxiliary X-ray image 22 having an array of sub-regions T of (e.g., identical) very small areas distributed over the detector surface.

As an alternative method to the method shown in FIG. 1, in act 15, a second setting of the collimator is made, and an auxiliary X-ray image 20 is acquired directly (see FIG. 2). The X-ray dose for acquiring the auxiliary X-ray image may be only a fraction of the X-ray dose of a conventional X-ray image, (e.g., 1/10 or less), in order to guarantee a minimum possible radiation load on the object under examination.

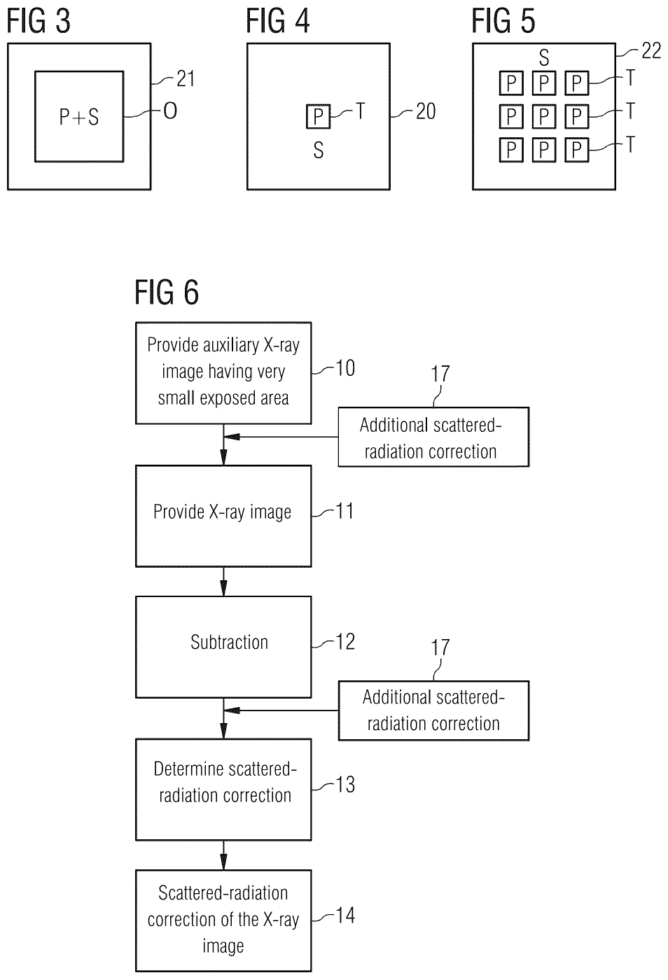

In act 11, an X-ray image 21 of the object under examination is provided (FIG. 1). Alternatively, in act 16, a first setting of the collimator is made, and the X-ray image is acquired directly (see FIG. 2). The X-ray image is, or was, acquired in a first setting of the collimator in such a way that an object under examination is illuminated, for example an organ, part of an organ, body part, or instrument situated in a hollow organ. The 2D X-ray image may also be part of a series of X-ray images, for example acquired successively for monitoring an interventional procedure or an examination, for instance during fluoroscopy. The first setting of the collimator in the acquisition of the 2D X-ray image 21 may illuminate an examination region O (see FIG. 3). The illuminated examination region O may include a significant portion of the total area of the X-ray detector, for instance at least ⅕ or ¼ of the total area of the X-ray detector. The first setting thus illuminates a far larger area with primary radiation than the second setting of the collimator. The image data of an X-ray image acquired in this manner has, in addition to the imaging component P produced by the primary radiation, an (e.g., unwanted) scattered radiation component S. In other words, the image data is composed of primary radiation component+scattered radiation component P+S.

Switching between the first setting and the second setting of the collimator and back may be performed very rapidly. It is performed as rapidly as possible in order to minimize interruption of the acquisition of X-ray images, for instance during live fluoroscopy. The order of acts 10 and 11, or acts 15 and 16, may be selected according to the application.

In act 12, the image data of the auxiliary X-ray image 20 is then subtracted from the image data of the 2D X-ray image 21, prior to which in particular a suitable adjustment may be made to account for X-ray parameters such as the X-ray dose (e.g. given an X-ray dose of the X-ray image that is 10 times that of the X-ray image, 10 times the value is subtracted). The subtraction is performed in particular pixel by pixel in accordance with known methods. It is also possible to perform a subtraction just in part, for instance excluding the image data in the region of the collimator shadow. In the region of the sub-region T of very small area there now results from the calculation P+S−P=S solely the scattered radiation component S. If a further (i.e. alternative) auxiliary X-ray image 22 is used, in which are illuminated a multiplicity of very small areas distributed over the total area, then in the region of the numerous sub-regions T may be identified a corresponding distribution of the scattered radiation S over the X-ray detector.

From this image data of the scattered radiation, in act 13, a scattered-radiation correction may be determined for the 2D X-ray image or for further similar 2D X-ray images (e.g., in a series of X-ray images). In one example, this may include deducting the scattered radiation component S in the sub-region, which is ascertained from the subtraction, from the image data of the acquired X-ray image (and/or further X-ray images of the series), for instance the same scattered radiation component S for each pixel. In such a case, the scattered-radiation correction would thus be the subtraction of an artificially produced subtraction mask, which subtracts the scattered radiation component S for each pixel of the X-ray image (or at least each pixel of the X-ray image in the directly exposed image region). Such a scattered-radiation correction may be accurate, for example, when the auxiliary X-ray image of the sub-region T used is located in the center of the X-ray detector.

It is also possible to use an algorithm to ascertain on the basis of the scattered radiation ascertained from the subtraction (act 12) a more complicated scattered-radiation correction that includes other factors such as X-ray dose, material composition of the object under examination, or angulation. In the case of a further auxiliary X-ray image that has a multiplicity of sub-regions distributed over the X-ray detector, the scattered-radiation correction may be ascertained as a subtraction mask in accordance with the ascertained scattered-radiation distribution. Regions between the sub-regions may be ascertained by interpolation or averaging or other methods. Image data from the collimator shadow may also be used to ascertain the scattered-radiation correction. In certain examples, it is also possible to use machine learning algorithms to ascertain a scattered-radiation correction.

In act 14, the X-ray image is corrected by the ascertained scattered-radiation correction, so for instance by subtracting the subtraction mask or applying an ascertained correction algorithm. It is also possible to correct a plurality of X-ray images or also a series of X-ray images by the ascertained scattered-radiation correction.

As soon as a (significant) change in X-ray parameters occurs, for instance X-ray dose, angulation, movement of the object under examination, etc., the method may be repeated, and the scattered-radiation correction then obtained thereby may be applied to the current X-ray image and/or further X-ray images of the series.

For the case that the scattered radiation in the sub-region affected by the primary radiation is not completely zero even for the auxiliary X-ray image, but just very small, an additional scattered-radiation correction may be performed before act 12. This is shown in FIG. 6. Here, after the acquisition/provision of the auxiliary X-ray image, in which the image data in the sub-region is composed of the primary radiation component P and a very small, scattered radiation component s, (i.e., R+s), is performed an additional scattered-radiation correction in act 17. For example, this scattered-radiation correction may be performed based on image data measured in the collimator shadow (which data arises solely from scattered radiation and, in the case of the auxiliary X-ray image, for example, is small), for instance by interpolation.

Furthermore, intermediate auxiliary X-ray images may also be provided and/or acquired that were acquired in a third and/or further setting(s) of the collimator. The corresponding sub-region(s) have an area that is larger than the very small area of the auxiliary X-ray image, but smaller than the illuminated area for the X-ray image. It is also possible here to acquire and/or provide a plurality of intermediate auxiliary X-ray images of increasing or decreasing size of the sub-regions (for instance each doubled or by gradual changes). In such a procedure, an increase in the scattered radiation component may be expected with an increase in the exposed area, which may be used for determining a scattered-radiation correction. For example, appropriate estimates, simulations or results ascertained by machine learning algorithms may be incorporated here. It is also possible to use a change in intensity for the sub-regions of different size in different intermediate auxiliary X-ray images, which sub-regions are irradiated using the same X-ray parameters each time, to increase the accuracy of the scattered-radiation estimate. With the additional use of the intermediate auxiliary X-ray images, a refined scattered-radiation correction may then be determined.

It may be possible to determine and use even more information about the scattered radiation. For example, the influence of the scattered radiation may be ascertained (e.g. calculated, interpolated, extrapolated or simulated) from the corresponding intermediate auxiliary X-ray images and the auxiliary X-ray image. The accuracy of the estimate of the scattered radiation may also be improved from the variation in the scattered radiation in the, for instance ever-smaller, collimator shadow and from the change in intensity for the region, which is irradiated equally each time.

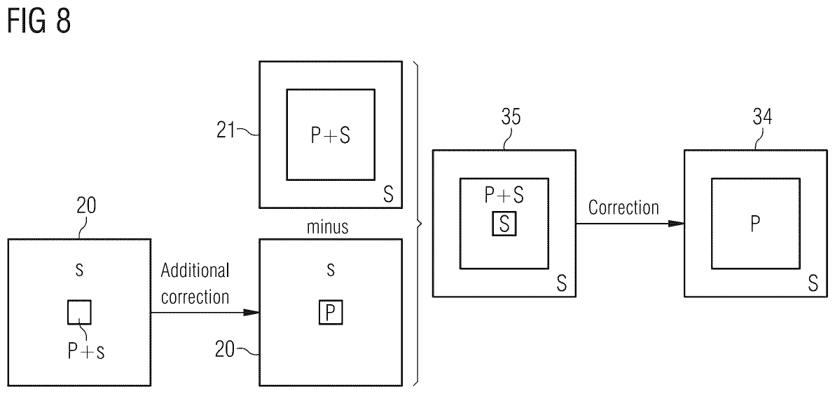

FIG. 8 shows the sequence of the method with reference to the respective X-ray images. Thus, it shows both the acquired X-ray image 21 containing the image data from primary radiation P and scattered radiation S, and the acquired auxiliary X-ray image 20 (either containing image data without scattered radiation or with low scattered radiation s, wherein the former may also be achieved by an additional correction). The subtraction results in a subtracted image 35. A corrected X-ray image 34 is obtained after performing the scattered-radiation correction.

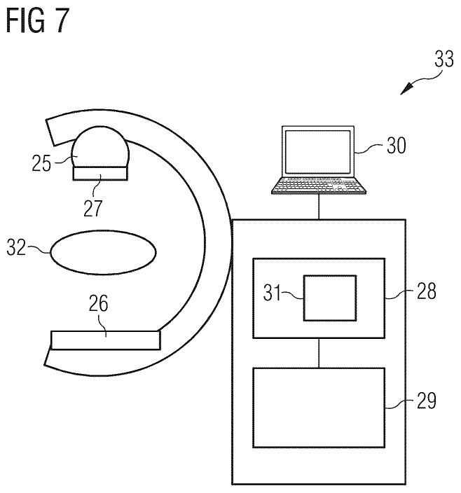

FIG. 7 shows an X-ray device 33 for performing the method for scattered-radiation correction. The X-ray device 33 has an acquisition system for acquiring X-ray images, having an X-ray detector 26, an X-ray source 25 for emitting primary X-ray radiation, and a collimator 27. The collimator is configured to shape the primary X-ray radiation and hence to illuminate sub-regions, which in particular have a multiplicity of conceivable sizes and positions, in particular also have a very small area. The collimator 27 may have for this purpose, for example, a large number of adjustable plates and/or shielding elements. A complex collimator having a multiplicity of asymmetrically positionable shielding elements may be used in particular for producing auxiliary X-ray images containing a multiplicity or array of sub-regions. The X-ray device 33 is controlled by a control unit 28, for instance to acquire X-ray images and to adjust the collimator into a first and second setting. The X-ray device also has a processing unit 29, which is configured for image processing and in particular for subtracting image data of the auxiliary X-ray image from the image data of the X-ray image. Subtraction methods are fundamentally known. In addition, the processing unit 29 and/or an additional computation unit is designed to determine a scattered-radiation correction using image data obtained from the subtraction at least in the region of the sub-regions, and to perform the scattered-radiation correction of X-ray images.

The disclosure may be summarized briefly as follows: for particularly rapid and effective scattered-radiation correction of 2D X-ray images, in particular a series of X-ray images, a method is provided for scattered-radiation correction for a (2D) X-ray image or a series of X-ray images, which images may be acquired by an acquisition system having an X-ray source, an X-ray detector, and a collimator that shapes the primary X-ray radiation. The method includes: providing the X-ray image, which was acquired in a first setting of the collimator, in which setting the object under examination is illuminated; providing an auxiliary X-ray image, which was acquired in a second setting of the collimator, in which second setting are illuminated in particular solely one or more sub-regions of the X-ray detector, each of which has a small area compared with the total area of the X-ray detector; subtracting the auxiliary X-ray image from the X-ray image; determining a scattered-radiation correction using image data from the X-ray detector obtained from the subtraction at least in the region of the sub-regions; and correcting the X-ray image using the determined scattered-radiation correction.

At the start of an X-ray scene, narrow collimation is performed for a single auxiliary X-ray image with a low dose. As a result of the narrow collimation, this auxiliary X-ray image then contains almost no scattered radiation. Subsequent enlargement of the collimator to the desired size results in scattered radiation being added to the image signal. Forming the difference then provides a very good estimate of the magnitude of the scattered radiation in the narrowly collimated sub-region. In addition, image information behind the collimator may also be used. The narrow collimation may be repeated if required, for instance if something in the acquisition situation has changed (e.g. patient movement, angulation change). This method may be repeated to any level of granularity.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for scattered-radiation correction of an X-ray image of an object under examination acquired by an acquisition system having an X-ray source, an X-ray detector, and a collimator that shapes a primary X-ray radiation, the method comprising:
providing an X-ray image, which was acquired in a first setting of the collimator in which the object under examination is illuminated;
providing an auxiliary X-ray image, which was acquired in a second setting of the collimator in which one or more sub-regions of the X-ray detector are illuminated, wherein each sub-region of the one or more sub-regions comprises an area that is less than a total area of the X-ray detector;
subtracting at least a part of the auxiliary X-ray image from the X-ray image;
determining a scattered-radiation correction using image data from the X-ray detector obtained from the subtraction at least in a region of the one or more sub-regions; and
correcting the X-ray image using the determined scattered-radiation correction.

2. The method of claim 1, wherein the one or more sub-regions comprise an overall area that is less than the total area of the X-ray detector.

3. The method of claim 1, further comprising, before the subtracting:
pre-correcting the auxiliary X-ray image for scattered radiation using image data from the X-ray detector obtained in a collimator shadow.

4. The method of claim 1, wherein the one or more sub-regions comprises a multiplicity of sub-regions.

5. The method of claim 4, wherein the multiplicity of sub-regions comprises an overall area that is less than the total area of the X-ray detector.

6. The method of claim 1, wherein a maximum area of each sub-region of the one or more sub-regions is 1/50 of the total area of the X-ray detector.

7. The method of claim 1, wherein an X-ray dose during the acquisition of the auxiliary X-ray image is at most 1/10 of an X-ray dose during the acquisition of the X-ray image.

8. The method of claim 1, wherein the X-ray image comprises a series of X-ray images acquired in succession.

9. The method of claim 1, further comprising:
providing at least one intermediate auxiliary X-ray image, which was acquired in a third setting of the collimator in which one or more sub-regions of the X-ray detector are illuminated, wherein each sub-region of the one or more sub-regions for the at least one intermediate auxiliary X-ray image has an area that is in between the area of the second setting and the total area of the X-ray detector,
wherein the intermediate auxiliary X-ray image is used to refine the determined scattered-radiation correction.

10. The method of claim 9, further comprising:
providing at least one further intermediate auxiliary X-ray image, which was acquired in further settings of the collimator in which at least one further sub-region is illuminated, wherein each sub-region of the at least one further sub-region has an area that is in between the area of the second setting and the total area of the X-ray detector,
wherein the at least one further intermediate auxiliary X-ray image is used to refine the determined scattered radiation.

11. The method of claim 1, wherein the X-ray image is a two-dimensional X-ray image.

12. A method for scattered-radiation correction of an X-ray image acquired by an acquisition system having an X-ray source, an X-ray detector, and a collimator that shapes a primary X-ray radiation, the method comprising:
setting a first setting of the collimator to illuminate an object under examination and acquiring an X-ray image by the acquisition system;
setting a second setting of the collimator to illuminate solely one or more sub-regions of the X-ray detector and acquiring an auxiliary X-ray image, wherein each sub-region of the one or more sub-regions has an area less than a total area of the X-ray detector;
subtracting at least a part of the auxiliary X-ray image from the X-ray image;
determining a scattered-radiation correction using image data from the X-ray detector obtained from the subtraction at least in a region of the one or more sub-regions; and
correcting the X-ray image using the determined scattered-radiation correction.

13. The method of claim 12, wherein the one or more sub-regions comprise an overall area that is less than the total area of the X-ray detector.

14. The method of claim 12, wherein a maximum area of each sub-region of the one or more sub-regions is 1/50 of the total area of the X-ray detector.

15. A medical X-ray device comprising:
an acquisition system configured to acquire an X-ray image and an auxiliary X-ray image, wherein the acquisition system comprises an X-ray detector, an X-ray source for emitting primary X-ray radiation, and a collimator that shapes the primary X-ray radiation, wherein the X-ray image is configured to be acquired in a first setting of the collimator in which an object under examination is illuminated, and wherein the auxiliary X-ray image is configured to be acquired in a second setting of the collimator in which one or more sub-regions of the X-ray detector are illuminated, wherein each sub-region of the one or more sub-regions comprises an area that is less than a total area of the X-ray detector;
a control unit configured to control the medical X-ray device;
a processing unit configured to:
subtract at least a part of the auxiliary X-ray image from the X-ray image;
determine a scattered-radiation correction using image data obtained from the subtraction at least in the region of the one or more sub-regions; and
perform a scattered-radiation correction of the X-ray image.
